(19) United States
(12) Patent Application Publication
(10) Pub. No.: US 2026/0350890 A1
(43) Pub. Date: Nov. 5, 2026

(54) DETECTOR SYSTEM AND CT SYSTEM

(71) Applicant: GE Precision Healthcare LLC, Waukesha, WI (US)

(72) Inventor: Yujiro Yazaki, Hino (JP)

(21) Appl. No.: 19/666,018

(22) Filed: May 1, 2026

(30) Foreign Application Priority Data
May 2, 2025 (JP) 2025-076702

(51) Int. Cl. G06T 12/10; A61B 6/03; A61B 6/42; A61B 6/58; G01T 1/17; G01T 7/00

(52) U.S. Cl. CPC G06T 12/10; A61B 6/032; A61B 6/4233; A61B 6/585; G01T 1/17; G01T 7/005; G06T 2210/41

(57) ABSTRACT

A detector system including a detector having a plurality of detector elements for detecting X-rays irradiated from an X-ray tube, and a member disposed between the X-ray tube and the detector and having a plurality of opening parts corresponding to the plurality of detector elements, the plurality of opening parts being opening parts through which the X-rays pass, and some of the opening parts among the plurality of opening parts having opening areas different from the remaining opening parts.

### CROSS-REFERENCE TO RELATED APPLICATIONS

This application claims priority to Japanese Application No. 2025-076702, filed on May 2, 2025, the disclosure of which is incorporated herein by reference in its entirety.

### TECHNICAL FIELD

The present invention relates to a detector system for detecting X-rays; and a CT system having the detector system.

### BACKGROUND

A CT system is known as a medical device that noninvasively images a subject. CT systems can acquire tomographic images of a subject in a short scanning time and therefore are widely used in hospitals and other medical facilities.

The CT system applies a prescribed voltage to a cathode-anode tube of an X-ray tube to generate X-rays. The generated X-rays penetrate the subject and are detected by a detector. The CT system reconstructs a CT image of the subject on the basis of data detected by the detector.

Furthermore, a CT image is desired to have high spatial resolution such that a fine structure of the imaging site can be accurately depicted. Therefore, various technologies have been developed to enhance spatial resolution. For example, a method is known in which the spatial resolution is improved by reducing the size of a detector element in an X-ray detector.

### SUMMARY OF THE INVENTION

However, this method requires the detector element to be reduced in size, which necessitates a complete redesign of the detector, resulting in increased development costs.

On the other hand, as a method for improving the spatial resolution, a method of arranging a member for improving the spatial resolution so as to face the detector is known. This member is configured such that openings are formed in regions corresponding to each of the detector elements of the detector, and the spatial resolution can be improved by reducing the opening area of the openings. However, while this method can improve spatial resolution, this method has the problem that the opening area is small, which reduces the efficiency of dose utilization and deteriorates contrast resolution. Therefore, a technology that achieves both spatial resolution and contrast resolution is desired.

A first aspect of the present invention is a detector system including a detector having a plurality of detector elements for detecting X-rays irradiated from an X-ray tube, and a member disposed between the X-ray tube and the detector and having a plurality of opening parts corresponding to the plurality of detector elements, the plurality of opening parts being opening parts through which the X-rays pass, and some of the plurality of opening parts having opening areas different from the remaining opening parts.

Furthermore, a second aspect of the present invention is a CT system including a detector system that includes a detector having a plurality of detector elements for detecting X-rays irradiated from an X-ray tube, and a member disposed between the X-ray tube and the detector and having a plurality of opening parts corresponding to the plurality of detector elements, the plurality of opening parts being opening parts through which the X-rays pass, and some of the plurality of opening parts having opening areas different from the remaining opening parts, the detector system being configured so as to generate a signal corresponding to each of the detector elements, wherein a CT image is reconstructed on the basis of the signal corresponding to each of the detector elements.

The member has opening parts with different opening areas. Therefore, a signal with high spatial resolution can be obtained from a detector element corresponding to a small opening area of the plurality of detector elements, while a signal with high contrast resolution can be obtained from a detector element corresponding to a large opening area. This facilitates achieving both high spatial resolution and high contrast resolution.

### BRIEF DESCRIPTION OF THE DRAWINGS

FIG. 1 is a block diagram of a CT system of an example embodiment.

FIG. 2 is a perspective view of a detector assembly of a comparative example.

FIG. 3 is an exploded perspective view of the detector assembly of the comparative example.

FIG. 4 is a top view of the detector assembly of the comparative example.

FIG. 5 is a perspective view of a detector assembly.

FIG. 6 is an exploded perspective view of the detector assembly.

FIG. 7 is a top view of the detector assembly.

FIG. 8 is a flowchart depicting an example of a method for processing signals obtained from the detector assembly.

FIG. 9 is an explanatory diagram of signals generated by a detector system.

FIG. 10 is a schematic diagram of a series of signals (separated into small opening signals and large opening signals.

FIG. 11 is an explanatory diagram of interpolation processing.

FIG. 12 is an explanatory diagram of step.

FIG. 13 is a flowchart depicting an example of a method for processing signals.

FIG. 14 is an explanatory diagram of signals generated by the detector system.

FIG. 15 is an explanatory diagram of a portion of the example method.

FIG. 16 is an explanatory diagram of a reason why noise reduction processing is executed on a small opening signal.

FIG. 17 is an explanatory diagram of a method for determining a reference signal for small opening signals.

FIG. 18 is an explanatory diagram of a portion of the example method.

FIG. 19 is a schematic diagram of an example of a lookup table.

FIG. 20 is a diagram depicting an example of a corrected small opening signal.

FIG. 21 is an explanatory diagram of a method for determining a reference signal for a large opening signal.

FIG. 22 is an explanatory diagram of a portion of the example method.

FIG. 23 is an explanatory diagram of the true value of a signal when Δ2<0.

FIG. 24 is an explanatory diagram of the true value of a signal when Δ2>0.

FIG. 25 is a diagram depicting an example of a corrected large opening signal.

FIG. 26 is a diagram depicting an example of a detector assembly in which a limiting member is disposed above a collimator.

FIG. 27 is a diagram depicting a modified example of the limiting member.

FIG. 28 is a diagram depicting another modified example of the limiting member.

FIG. 29 is a diagram depicting an example of a detector assembly including a member having both the functions of a limiting member and a collimator.

### DETAILED DESCRIPTION OF THE DRAWINGS

Embodiments for carrying out the invention will be described below, but the present invention is not limited to the following embodiments.

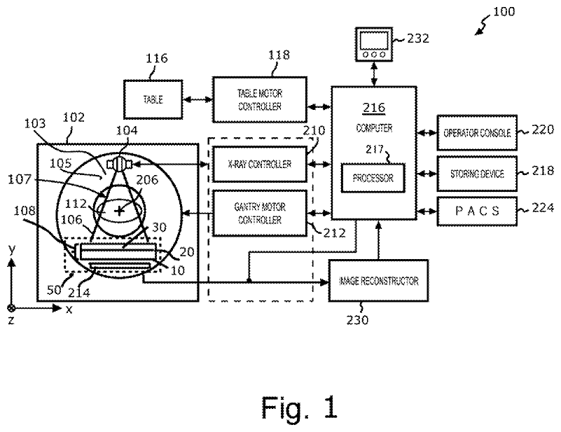

FIG. 1 is a block diagram of a CT system 100 of an example embodiment. The CT system 100 includes a gantry 102 and a table 116. The gantry 102 has a bore 107. The table 116 supports a subject 112 and moves the subject 112 into the bore 107. An X-ray tube 104, a filter part 103, a pre-collimator 105, a detector system 50, and the like are attached to the gantry 102.

The X-ray tube 104 outputs X-rays 106 when a prescribed voltage is applied to a cathode-anode tube. The X-ray tube 104 is configured to be rotatable on a path centered on a rotation axis 206 within the XY plane. Herein, the Z direction represents the body-axis direction, the Y direction represents the vertical direction (the height direction of the table 116), and the X direction represents the direction perpendicular to the Z and Y directions. The X-ray tube 104 may be an X-ray tube compatible with single-energy CT or an X-ray tube compatible with dual-energy CT.

The filter part 103 includes, for example, a flat plate filter and/or a bow-tie filter, or the like. The pre-collimator 105 is a member that narrows the X-ray irradiation range such that X-rays are not irradiated in unwanted regions. The detector system 50 has a detector assembly 108 and a DAS 214. The detector assembly 108 has a detector 10, a limiting member 20, and a collimator 30. The limiting member 20 is overlaid on the detector 10, and the collimator 30 is overlaid on the limiting member 20. Therefore, the detector assembly 108 is arranged in the following order when viewed from the X-ray tube 104: the collimator 30, the limiting member 20, and the detector 10. In other words, the limiting member 20 is disposed between the X-ray tube 104 and the detector 10. The limiting member 20 is a member for limiting a region through which X-rays can pass. The limiting member 20 will be described later in detail.

The detector 10 has a plurality of detector elements that detect X-rays. The detector 10 generates a signal corresponding to the detected X-rays and outputs the signal to the DAS 214. Note that the detector 10 may be an energy-integrating detector or a photon-counting detector, and an arbitrary type of detector can be used. The DAS 214 executes prescribed processing, including sampling, digital conversion, and the like, on the signal received from the detector 10. The processed signal is transmitted to a computer 216. The computer 216 processes the signal received from the DAS 214. The computer 216 has a storing device 218 linked thereto. The storing device 218 includes one or more storing medium that records a program, instruction, and the like to be executed by the processor. The storing medium may be, for example, one or more non-transitory computer-readable storing medium. The storing device 218 may include, for example, hard disk drives, floppy disk drives, compact disc read/write (CD-R/W) drives, digital versatile disk (DVD) drives, flash drives, and/or solid state recording drives.

The computer 216 includes one or a plurality of processors 217. The computer 216 uses the one or plurality of processors 217 to output commands and parameters to the DAS 214, X-ray controller 210, and/or gantry motor controller 212, to control system operations such as data acquisition and/or processing. Furthermore, the computer 216 can use the one or plurality of processors 217 to execute various processes such as signal processing, data processing, and image processing, in each step of the flow (to be described later). The processor 217 may include one or a plurality of general-purpose processors, one or a plurality of application specific integrated circuits, one or a plurality of field programmable gate arrays, or the like. Note that in FIG. 1, the one or plurality of the processors 217 are included in the computer 216, but the one or plurality of the processors 217 may be provided so as to be distributed between the computer 216 and another component (e.g., image reconstructor 230).

An operator console 220 is linked to the computer 216. An operator can enter prescribed operator inputs related to an operation of the CT system 100 into the computer 216 by operating the operator console 220. The computer 216 receives an operator input, including a command and/or scan parameter, via the operator console 220 and controls system operation on the basis of the operator input. The operator console 220 can include a keyboard (not depicted) or touch screen for the operator to specify a command and/or scan parameter.

The X-ray controller 210 controls the X-ray tube 104 on the basis of an instruction from the computer 216. Furthermore, the gantry motor controller 212 controls a gantry motor to rotate a component, such as the X-ray tube 104, detector system 50, or the like, on the basis of an instruction from the computer 216.

FIG. 1 depicts only one operator console 220, but two or more operator consoles may be linked to the computer 216. Furthermore, the CT system 100 may also allow a plurality of remotely located displays, printers, workstations, and/or similar devices to be linked via, for example, a wired and/or wireless network. In one embodiment, for example, the CT system 100 may include a Picture Archiving and Communication System (PACS) 224, or may be linked to the PACS 224. In a typical implementation, a PACS 224 may be linked to a remote system such as a radiology department information system, hospital information system, and/or an internal or external network (not depicted) or the like.

The computer 216 provides an instruction to a table motor controller 118 to control the table 116. The table motor controller 118 can control the table motor so as to move the table 116 on the basis of the instructions received. For example, the table motor controller 118 can move the table 116 such that the subject 112 is positioned appropriately for imaging.

The signal processed by the DAS 214 is also transmitted to an image reconstructor 230. The image reconstructor 230 reconstructs a CT image on the basis of the signal received from the DAS 214. The image reconstructor 230 includes one or a plurality of processors, which can execute image reconstruction processing. In FIG. 1, the image reconstructor 230 is depicted as a separate component from the computer 216, but the image reconstructor 230 may form a part of the computer 216. Furthermore, the computer 216 may also perform one or a plurality of functions of the image reconstructor 230. Furthermore, the image reconstructor 230 may be positioned away from the CT system 100 and operatively connected to the CT system 100 using a wired or wireless network.

The image reconstructor 230 can store the reconstructed image in the storing device 218. The image reconstructor 230 may also transmit the reconstructed image to the computer 216. The computer 216 can transmit the reconstructed image and/or patient information to a display device 232 communicatively linked to the computer 216 and/or image reconstructor 230.

Non-transitory computer-readable storing medium included in the CT system 100 or in communication with the CT system 100 may store instructions for executing the various methods, steps, and processes described in the present specification. The instructions may be stored on a single storing medium or distributed across a plurality of storing media. Furthermore, the instructions may be stored on an external storing device accessible by the CT system. One or more processors provided in the CT system 100 execute the various methods, steps, and processes described in the present specifications in accordance with the instructions recorded on the storing medium. The CT system 100 is configured as described above.

In one embodiment, a CT image of a subject can be reconstructed using the CT system configured as described above. A physician (e.g., a radiologist) uses the CT image to diagnose a patient. Therefore, in order to improve diagnostic accuracy, a CT image with high spatial resolution is desired. For example, a method is known in which the spatial resolution is improved by reducing the size of a detector element in an X-ray detector. However, this method requires the detector element to be reduced in size, which necessitates a complete redesign of the detector, resulting in increased development costs.

On the other hand, as a method for improving the spatial resolution, a method of arranging a member for improving the spatial resolution so as to face the detector is known. This member is configured such that openings are formed in regions corresponding to each of the detector elements of the detector, and the spatial resolution can be improved by reducing the opening area of the openings. However, while this method can improve spatial resolution, this method has the problem that the opening area is small, which reduces the efficiency of dose utilization and deteriorates contrast resolution.

Therefore, in one example embodiment, the detector assembly 108 is configured to achieve both spatial resolution and contrast resolution. Hereinafter, the detector assembly 108 of an example embodiment will be described.

Note that in describing an example embodiment, an example of a detector assembly of a comparative example, which can improve spatial resolution but not achieve both spatial resolution and contrast resolution will be briefly described in order to facilitate ease of understanding of the effects of the example embodiment. Furthermore, after describing the detector assembly of the comparative example, the detector assembly 108 in an example embodiment, which can achieve both high spatial resolution and high contrast resolution, will be described.

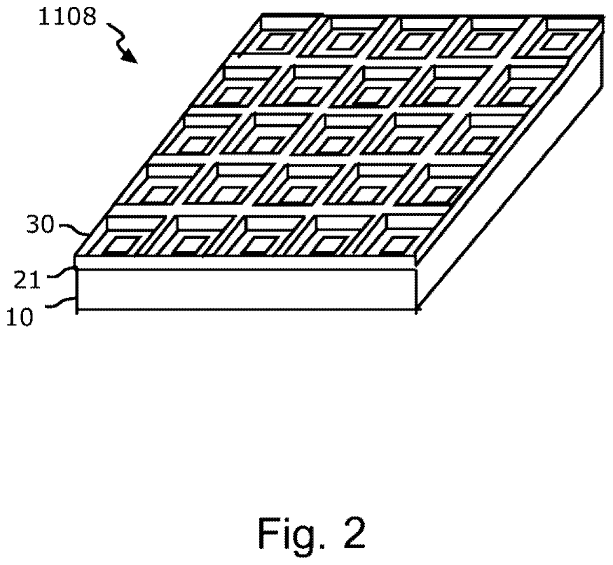

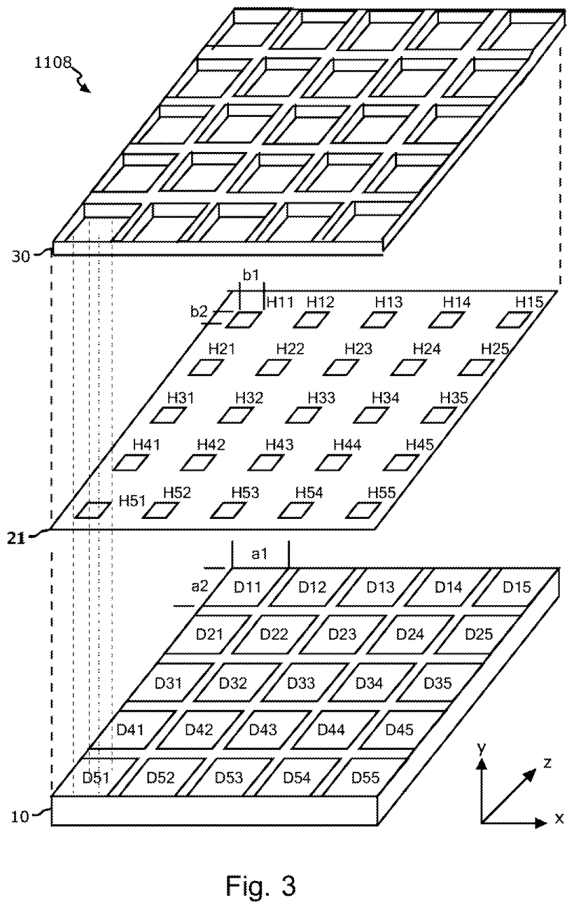

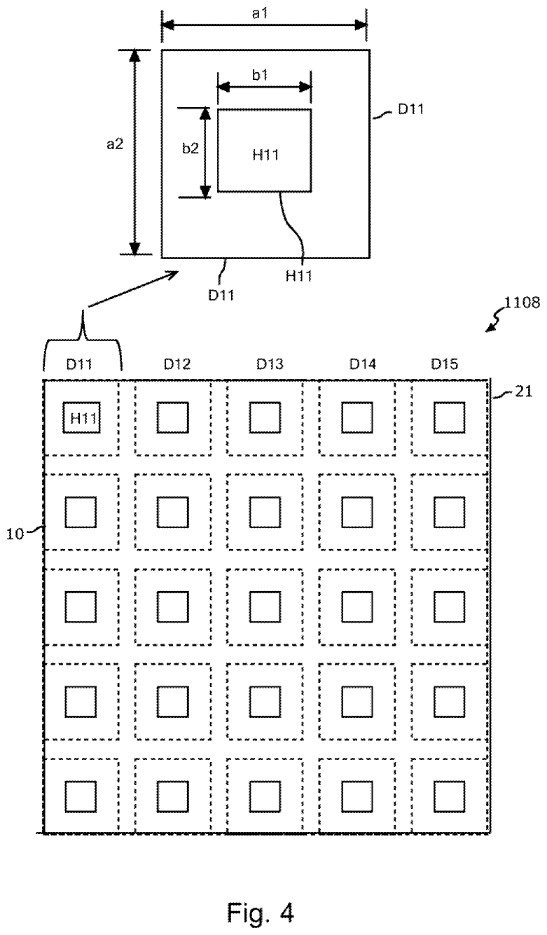

FIG. 2 is a perspective view of the detector assembly 1108 of the comparative example, FIG. 3 is an exploded perspective view of the detector assembly 1108 of the comparative example, and FIG. 4 is a top view of the detector assembly 1108 of the comparative example. In FIGS. 2 to 4, in order to make the configuration of detector assembly 1108 easier to see, the number of detector elements in the detector assembly 1108 is depicted as being less than the actual number of detector elements, and FIGS. 2 to 4 depict five rows and five columns of detector elements. Note that in FIG. 4, the collimator 30 is omitted in order to make the detector 10 and a limiting member 21 easier to see.

The detector assembly 1108 of the comparative example includes the detector 10, a limiting member 21 for limiting the region through which X-rays can pass, and the collimator 30.

As depicted in FIG. 3, the detector 10 has detector elements D11 to D55 arranged in five rows and five columns. Furthermore, FIG. 3 (FIG. 4) depicts a length a1 of the detector element D11 in the channel direction and a length a2 in the body axis direction (z direction). The length a1 may be the same value as the length a2 (a1=a2), or the length a1 may be a different value from the length a2 (a1≠a2). Note that the other detector elements D12 to D55 are also configured to have the same dimensions (lengths a1 and a2) as the detector element D11. The detector 10 also has partition wall for separating adjacent detector elements. The partition walls are provided for the purpose of preventing crosstalk and the like between adjacent detectors 10.

Furthermore, the detector assembly 1108 of the comparative example includes the collimator 30. The collimator 30 is provided in order to prevent scattering of the X-rays irradiated from the X-ray tube, and has a lattice shape. Furthermore, the detector assembly 1108 of the comparative example has the limiting member 21 disposed between the detector 10 and the collimator 30. The limiting member 21 has opening parts H11 to H55 at positions corresponding to the detector elements. Furthermore, FIG. 3 (FIG. 4) depicts a length b1 of the opening part H11 in the channel direction and a length b2 in the body axis direction (z direction). The opening part H11 is formed such that the length b1 is the same as the length b2 (b1=b2). Note that the other opening parts H12 to H55 are also configured to have the same dimensions (lengths b1 and b2) as the opening part H11.

The detector assembly 1108 of the comparative example is configured such that the opening area of the opening part is a value that is smaller than the area of the detector element, thereby improving spatial resolution. However, while the detector assembly 1108 of the comparative example can improve spatial resolution, the detector assembly 1108 has a problem that the opening area is small, which reduces the efficiency of dose utilization and deteriorates contrast resolution.

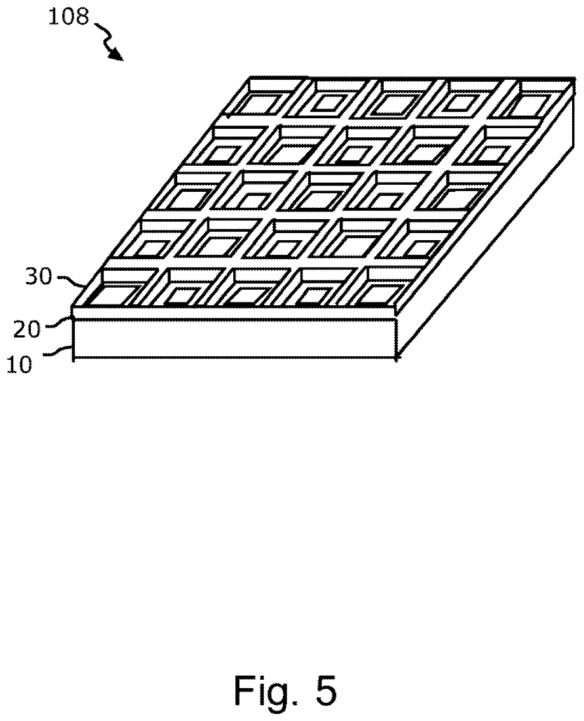

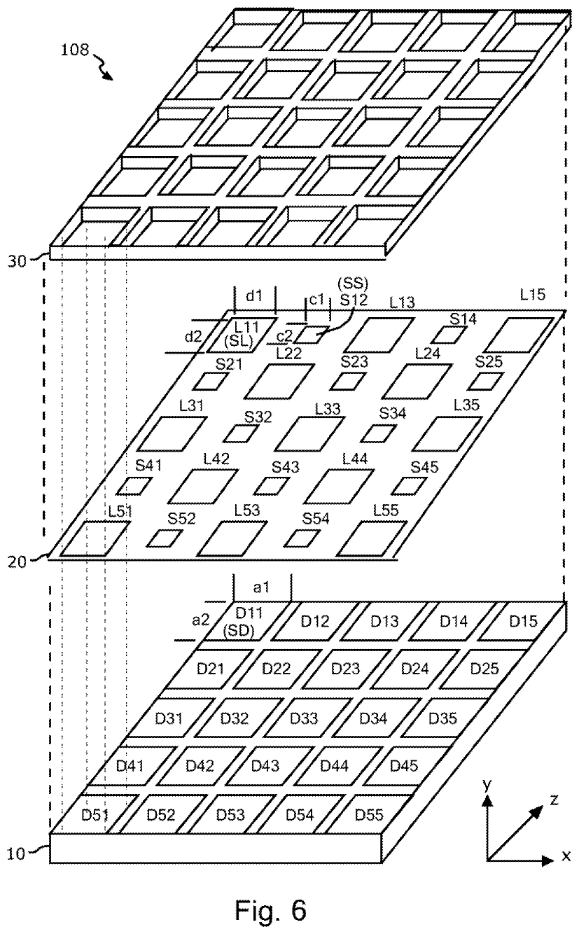

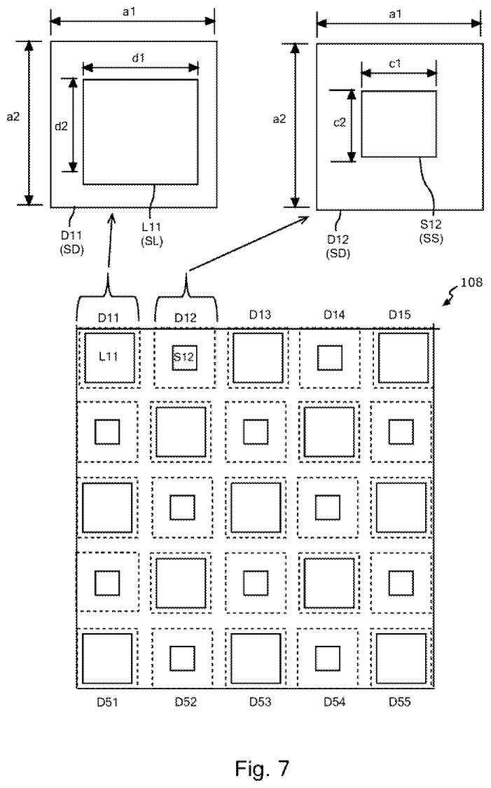

Therefore, in an example embodiment, the detector assembly 108 is configured to achieve both spatial resolution and contrast resolution. Hereinafter, the detector assembly 108 of the example will be described. FIG. 5 is a perspective view of the detector assembly 108 of the example, FIG. 6 is an exploded perspective view of the detector assembly 108 of the example, and FIG. 7 is a top view of the detector assembly 108 of the example.

The detector assembly 108 of the example includes the detector 10, the limiting member 20 for limiting the region through which X-rays can pass, and the collimator 30. When viewing the detector assembly 108 from the X-ray tube 104 (see FIG. 1), the collimator 30 is positioned closest to the X-ray tube 104, the limiting member 20 is positioned second closest to the X-ray tube 104, and the detector 10 is positioned furthest from the X-ray tube 104.

Note that the detector 10 and the collimator 30 of the detector assembly 108 of the example have the same structures as the detector 10 and the collimator 30 of the detector assembly 1108 of the comparative example. Therefore, in describing the detector assembly 108 of the example, the limiting member 20 will be mainly described.

The limiting member 20 is disposed between the detector 10 and the collimator 30. The limiting member 20 is a member formed in a sheet-like shape, and can be made using a material such as molybdenum or tungsten. The thickness of the limiting member 20 can be, for example, less than 1 mm, but can also be 1 mm or more if necessary. The limiting member (opening member) 20 is configured to demarcate a plurality of opening parts. Specifically, the limiting member 20 has a plurality of opening parts L11 to L55 and S12 to S54 for allowing X-rays to pass at positions corresponding to the detector elements. In the example, as a feature of the limiting member 20, some of the plurality of opening parts are formed to have opening areas different from the remaining opening parts. Specifically, the limiting member 20 includes an opening part having a small opening area and an opening part having a large opening area. In FIG. 6, the opening parts with small opening areas and the opening parts with large opening areas are as follows:
(1) Opening parts with small opening areas: S12, S14, S21, S23, S25, S32, S34, S41, S43, S45, S52, and S54
(2) Opening parts with large opening areas: L11, L13, L15, L22, L24, L31, L33, L35, L42, L44, L51, L53, and L55

Hereinafter, for convenience of explanation, an opening part having a small opening area will be referred to as a “small opening part”, and an opening part having a large opening area will be referred to as a “large opening part”.

First, the small opening part will be described. The small opening part S12 has a length c1 in the channel direction and a length c2 in the body axis direction (see FIGS. 6 and 7). Note that the remaining small opening parts S14 to S54 also have the length c1 in the channel direction and the length c2 in the body axis direction, similar to the small opening part S12. Therefore, the small opening parts S12 to S54 have the same opening area SS. The opening area SS of the small opening parts S12 to S54 is set to a value smaller than an area SD of the detector element. The opening area SS of the small opening part can be set to a value within the range from 10% to 80% of the area SD of the detector element, for example.

Next, the large opening parts L11 to L55 will be described. The large opening part L11 has a length d1 in the channel direction and a length d2 in the body axis direction. Similarly to the large opening part L11, the remaining large opening parts L13 to L55 each have the length d1 in the channel direction and the length d2 in the body axis direction. Therefore, the large opening parts L11 to L55 have the same opening area SL.

A feature of the large opening parts L11 to L55 is that these large opening parts have larger opening areas than the small opening parts S12 to S54. In other words, the opening area SL of the large opening part is set to be larger than the opening area SS of the small opening part (SL>SS). For example, the opening area SL of the large opening part can be set to twice the opening area SS of the small opening part. Note that the opening area SL of the large opening part may be smaller or larger than the area SD of the detector element, as long the condition is satisfied in which the opening area SL is larger than the opening area SS of the small opening part. The limiting member 20 is configured as described above.

Therefore, the small opening parts and large opening parts of the limiting member 20 allow X-rays to pass, but other regions of the limiting member 20 (i.e., regions other than the small opening parts and large opening parts) are configured not to allow X-rays to pass. Therefore, the limiting member 20 can limit the region through which X-rays can pass.

In the example, large opening parts are arranged on both sides of small opening parts in the channel direction, and large opening parts are arranged on both sides of small opening parts in the body axis direction (z direction). Therefore, the small opening parts are arranged such that at least two large opening parts are adjacent to each other, and the large opening parts are arranged such that at least two small opening parts are adjacent to each other.

The detector 10 includes a plurality of first detector elements corresponding to the plurality of small opening parts and a plurality of second detector elements corresponding to the plurality of large opening parts. Furthermore, the detector system 50 uses a first detector element corresponding to a small opening part to generate a signal corresponding to the first detector element (hereinafter referred to as a “small opening signal”), and uses a second detector element corresponding to a large opening part to generate a signal corresponding to the second detector element (hereinafter referred to as a “large opening signal”).

As described above, the limiting member 20 of the example embodiment has both a small opening part and a large opening part. The reason for providing both the small opening parts and the large opening parts will be described hereinafter.

X-rays irradiated from the X-ray tube 104 are reflected and scattered by a human body and the CT system (gantry, table, and the like) before reaching the detector elements, and therefore, the detector elements receive X-rays incident from various directions. However, the opening area of the small opening part is small, and therefore, incident directions of X-rays that can be received by the detector element can be limited. Therefore, the detector system 50 can output a signal with high spatial resolution through the small opening part. However, the small opening part has a problem in that the efficiency of X-ray dose utilization is reduced due to the small opening area thereof, resulting in a reduction in the contrast resolution.

On the other hand, the large opening part has a large opening area, and thus cannot effectively limit the incident directions of X-rays that can be received by the detector elements, as compared with the small opening part, thereby reducing the spatial resolution. However, the large opening part has a large opening area, and thus can improve the efficiency of X-ray dose utilization, resulting in an improvement in the contrast resolution.

Therefore, the detector system 50 can generate a signal from X-rays that pass through the small opening part that contribute to improved spatial resolution, while generating a signal from X-rays that pass through the large opening part that contribute to improved contrast resolution. Therefore, by including the detector assembly 108 configured as described above, the detector system 50 can achieve both spatial resolution and contrast resolution.

Next, a description will be provided on how signals obtained from the detector assembly 108 configured as described above are processed to achieve both spatial resolution and contrast resolution.

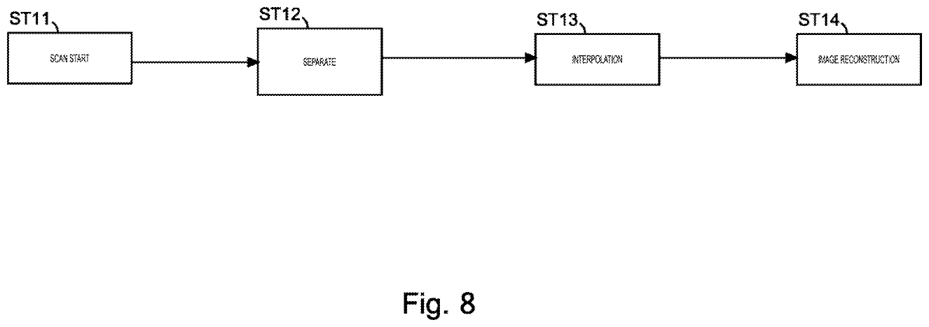

FIG. 8 is a flow depicting an example of a method for processing a signal obtained from the detector assembly 108 of an example embodiment.

In step ST11, scanning is started. When scanning is started, X-rays are irradiated from the X-ray tube 104. The X-rays pass through the subject and are detected by the detector system 50. The detector system 50 generates a signal in response to the detected X-rays.

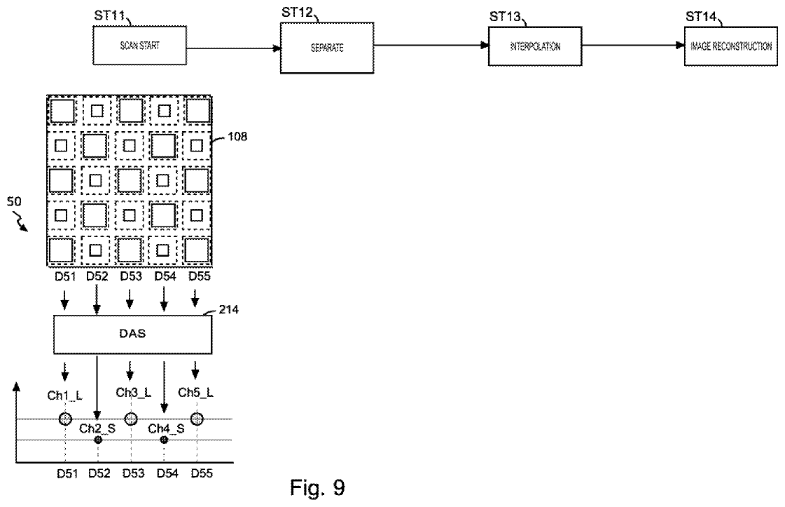

FIG. 9 is an explanatory diagram of signals generated by the detector system 50. FIG. 9 schematically depicts the signals generated by the detector system 50. The detector system 50 detects the X-rays using the detector assembly 108. The detector assembly 108 generates a signal corresponding to the detected X-rays. The signal from the detector assembly 108 is output to the DAS 214. The DAS 214 receives the signal, processes the signal, and generates a signal corresponding to each detector element. Herein, for convenience of description, a situation is depicted in which signals Ch1_L, Ch2_S, Ch3_L, Ch4_S, and Ch5_L are generated corresponding to the five detector elements D51, D52, D53, D54, and D55. The signal generated by the DAS 214 is output to the computer 216.

In FIG. 9, large opening signals (Ch1_L, Ch3_L, Ch5_L) corresponding to the detector elements for the large opening parts are indicated by large circles, while small opening signals (Ch2_S, Ch4_S) corresponding to the detector elements for the small opening parts are indicated by small circles.

Note that the large opening signals and small opening signals actually have signal values corresponding to each tissue of the subject, but for convenience of explanation, the large opening signals (Ch1_L, Ch3_L, Ch5_L) are considered to have the same signal value, and the small opening signals (Ch2_S, Ch4_S) are also considered to have the same signal value. After the large opening signals (Ch1_L, Ch3_L, Ch5_L) and small opening signals (Ch2_S, Ch4_S) are generated, the process proceeds to step ST12.

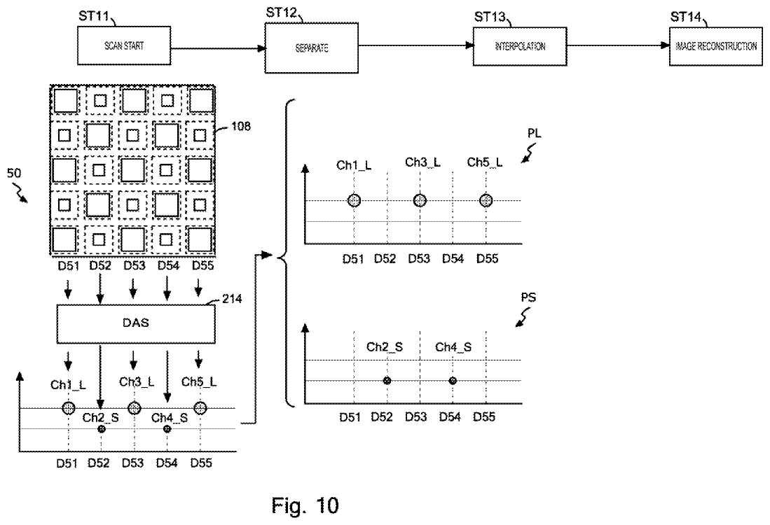

In step ST12, the computer 216 uses one or a plurality of processors to separate a series of the signals (Ch1_L, Ch2_S, Ch3_L, Ch4_S, Ch5_L) received from the DAS 214 into the small opening signals and large opening signals (see FIG. 10).

FIG. 10 is a schematic diagram of the series of the signals (Ch1_L, Ch2_S, Ch3_L, Ch4_S, Ch5_L) separated into the small opening signals and large opening signals.

The processor separates the series of the signals (Ch1_L, Ch2_S, Ch3_L, Ch4_S, Ch5_L) into the small opening signals and large opening signals. Therefore, two sets of projection data (projection data PS containing the small opening signals and projection data PL containing the large opening signals) can be generated. In FIG. 10, the signals (Ch1_L, Ch2_S, Ch3_L, Ch4_S, Ch5_L) corresponding to the five detector elements D51 to D55 are used as an example for creating two sets of projection data, but similarly, two sets of projection data can be created for signals from other detector elements. After the signals are separated, the process proceeds to step ST13.

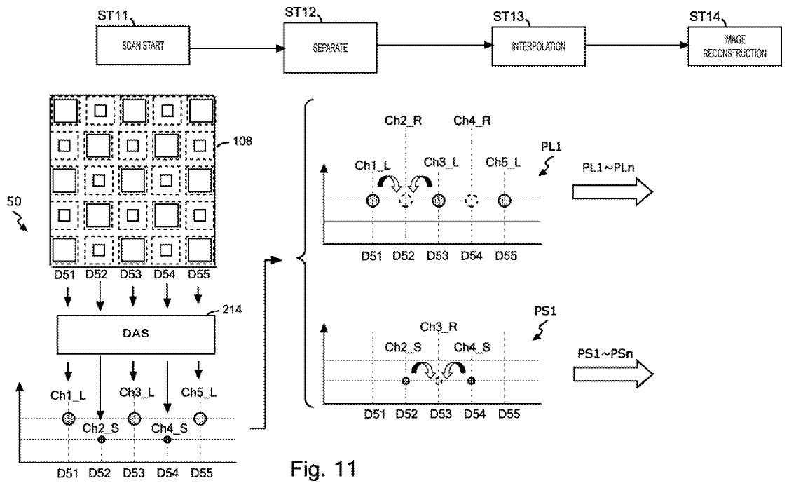

In step ST13, the computer 216 uses the one or plurality of processors to execute interpolation processing on the two sets of projection data PS and PL obtained in step ST12 to interpolate data points at points where data is missing (see FIG. 11).

FIG. 11 is an explanatory diagram of interpolation processing. The processor executes interpolation processing on the projection data PL to interpolate data points of the detector element D52 and D54. Specifically, the processor uses the signals Ch1_L and Ch3_L from the projection data PL to interpolate a data point for the detector element D52, and generate an interpolation signal Ch2_R for the detector element D52. Furthermore, the processor uses the signals Ch3_L and Ch5_L of the projection data PL to interpolate a data point for the detector element D54, and generate an interpolation signal Ch4_R for the detector element D54. Therefore, interpolated projection data PL1 can be generated.

Furthermore, the processor executes interpolation processing on the projection data PS to interpolate a data point of the detector element D53. Specifically, the processor uses the signals Ch2_S and Ch4_S from the projection data PS to interpolate a data point for the detector element D53, and generate an interpolation signal Ch3_R for the detector element D53. Therefore, interpolated projection data PS1 can be generated. Note that in an example embodiment, data points are interpolated using signals in the channel direction, but data points may also be interpolated using signals in the body axis direction (column direction) or signals acquired from another view.

Note that, while FIG. 9 to FIG. 11 describe signal processing for a series of signals (Ch1_L, Ch2_S, Ch3_L, Ch4_S, Ch5_L), another series of signals may also be separated into the two sets of projection data PL and PS and interpolated. Therefore, a plurality of sets of interpolated projection data PL1 to PLn and a plurality of sets of interpolated projection data PS1 to PSn can be obtained. After interpolation processing is executed, the process proceeds to step ST14.

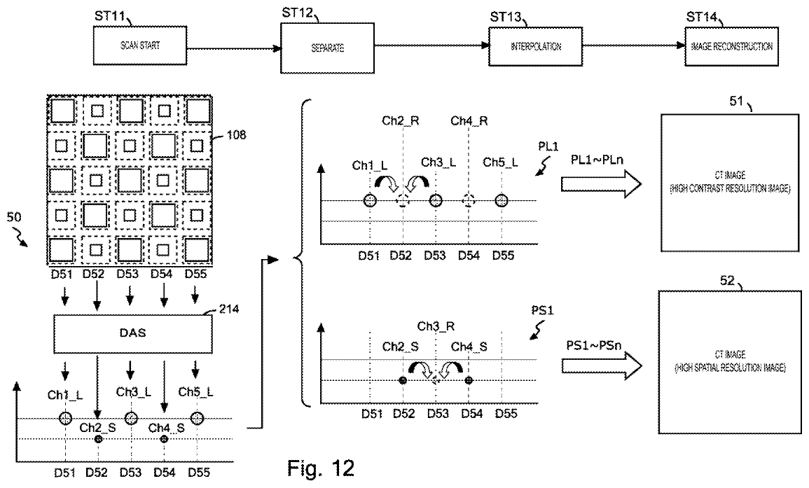

FIG. 12 is an explanatory diagram of step ST14. In step ST14, the image reconstructor 230 reconstructs a CT image 51 on the basis of the plurality of sets of interpolated projection data PL1 to PLn. The interpolated projection data PL1 to PLn is data obtained from signals of the detector elements corresponding to the large opening parts. Therefore, by reconstructing the image on the basis of the interpolated projection data PL1 to PLn, a CT image with high contrast resolution can be obtained.

Furthermore, the image reconstructor 230 reconstructs a CT image 52 on the basis of the plurality of sets of interpolated projection data PS1 to PSn. The interpolated projection data PS1 to PSn is data obtained from signals of the detector elements corresponding to the small opening parts. Therefore, by reconstructing the image on the basis of the interpolated projection data PS1 to PSn, a CT image with high spatial resolution can be obtained.

As described above, in an example, the detector assembly 108 is provided with a limiting member that has both the small opening parts and large opening parts. Therefore, a small opening signal with high spatial resolution can be obtained from a small opening part, and a large opening signal with high contrast resolution can be obtained from a large opening part. Therefore, the CT image 51 with high contrast resolution is reconstructed on the basis of the large opening signal, and the CT image 52 with high spatial resolution is reconstructed on the basis of the small opening signal. Therefore, a physician can diagnose the subject on the basis of both the CT image 51 with high contrast resolution and the CT image 52 with high spatial resolution, thereby improving the accuracy of the diagnosis.

In an example, by providing the limiting member 20 between the detector 10 and the collimator 30, both a small opening signal and large opening signal can be obtained. Therefore, even without manufacturing a new detector with a special structure in which small detector elements and large detector elements are alternately arranged, small opening signals and large opening signals can be generated by combining the limiting member 20 with an existing detector and limiting member. As a result, an existing detector can be used as is, and therefore, there is no need to design a new detector from scratch with a special structure in which small detector elements and large detector elements are alternately arranged, thereby avoiding an increase in detector manufacturing costs.

Note that in an example, the CT image 51 is reconstructed on the basis of the plurality of sets of interpolated projection data PL1 to PLn, and the CT image 52 is reconstructed on the basis of the plurality of sets of interpolated projection data PS1 to PSn. However, data blending processing that combines the plurality of sets of interpolated projection data PL1 to PLn and the plurality of sets of interpolated projection data PS1 to PSn can also be executed to reconstruct the CT images 51 and 52.

In another example, an example was described in which the series of signals (Ch1_L, Ch2_S, Ch3_L, Ch4_S, Ch5_L) are separated into the small opening signals and the large opening signals, and the CT image 51 with high contrast resolution and the CT image 52 with high spatial resolution are reconstructed as separate CT images. In an example embodiment, an example will be described in which a CT image is reconstructed that simultaneously achieves both spatial resolution and contrast resolution on the basis of the series of signals (Ch1_L, Ch2_S, Ch3_L, Ch4_S, Ch5_L).

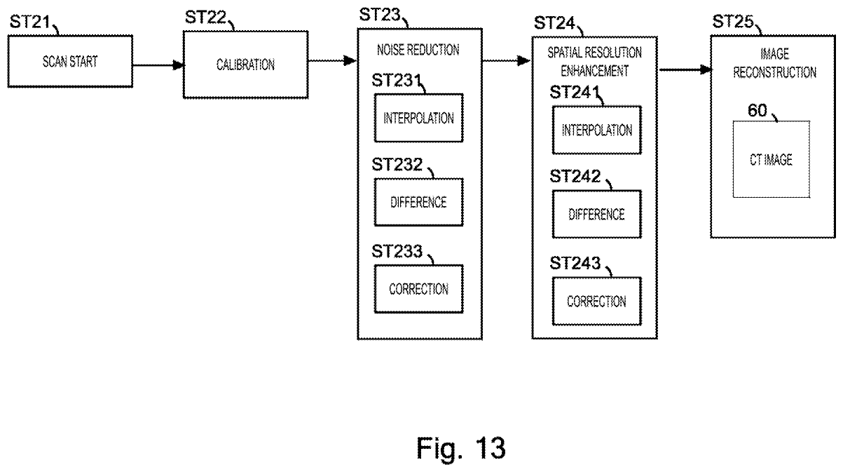

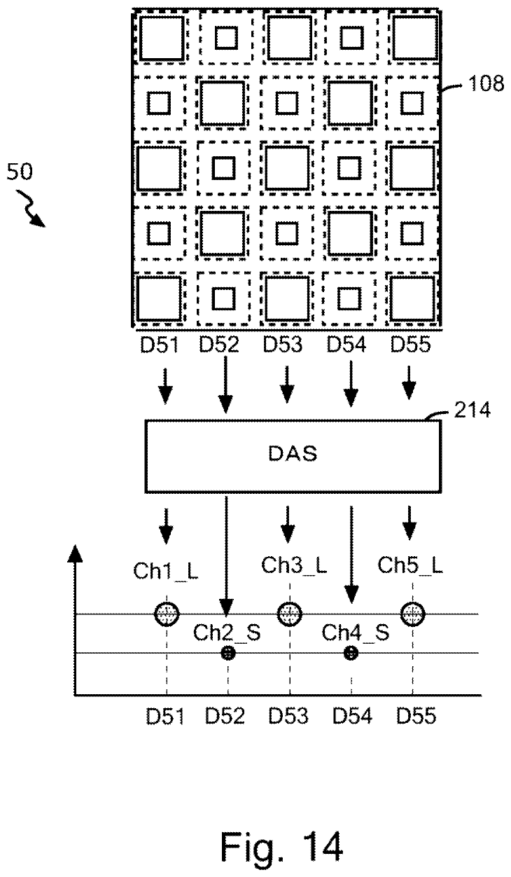

FIG. 13 is a flowchart depicting an example of a method for processing signals according to an example embodiment. In step ST21, scanning is started. When scanning is started, X-rays are irradiated from the X-ray tube 104. The X-rays pass through the subject and are detected by the detector system 50. The detector system 50 generates a signal in response to the detected X-rays (see FIG. 14).

FIG. 14 is an explanatory diagram of signals generated by the detector system 50. FIG. 14 schematically depicts the signals generated by the detector system 50. The detector system 50 detects the X-rays using the detector assembly 108. The detector assembly 108 generates a signal corresponding to the detected X-rays. The signals from the detector assembly 108 are processed by the DAS 214 to generate signals corresponding to each detector element. Herein, for convenience of description, a situation is depicted in which signals Ch1_L, Ch2_S, Ch3_L, Ch4_S, and Ch5_L are generated corresponding to the five detector elements D51, D52, D53, D54, and D55. The signal generated by the DAS 214 is output to the computer 216.

In FIG. 14, large opening signals (Ch1_L, Ch3_L, Ch5_L) corresponding to the detector elements for the large opening parts are indicated by large circles, while small opening signals (Ch2_S, Ch4_S) corresponding to the detector elements for the small opening parts are indicated by small circles.

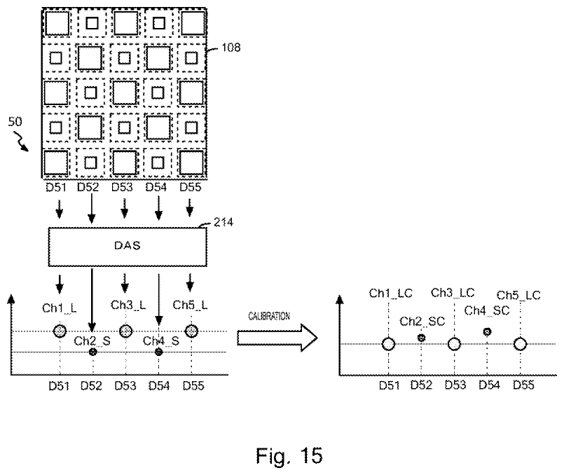

Note that the large opening signals and small opening signals actually have signal values corresponding to each tissue of the subject, but for convenience of explanation, the large opening signals (Ch1_L, Ch3_L, Ch5_L) are considered to have the same signal value, and the small opening signals (Ch2_S, Ch4_S) are also considered to have the same signal value. After the large opening signals (Ch1_L, Ch3_L, Ch5_L) and small opening signals (Ch2_S, Ch4_S) are generated, the process proceeds to step ST22 (see FIG. 15).

FIG. 15 is an explanatory diagram of step ST22. In step ST22, the computer 216 executes calibration of a series of the signals (Ch1_L, Ch2_S, Ch3_L, Ch4_S, Ch5_L) using one or a plurality of processors. The limiting member 20 used in the detector assembly 108 (e.g., see FIG. 6) has opening parts with different opening areas. Therefore, in order to calibrate signal value deviations due to differences in the opening area, calibration of the series of signals (Ch1_L, Ch2_S, Ch3_L, Ch4_S, Ch5_L) is executed on the basis of calibration data. Note that the calibration data is data acquired in advance before executing scanning of the subject, and is stored in a storing device. The processor can execute calibration of the series of signals (Ch1_L, Ch2_S, Ch3_L, Ch4_S, Ch5_L) using the calibration data stored in the storing device. In FIG. 15, the calibrated signals obtained by calibrating the signals (Ch1_L, Ch2_S, Ch3_L, Ch4_S, Ch5_L) are indicated as (Ch1_LC, Ch2_SC, Ch3_LC, Ch4_SC, Ch5_LC). After calibration processing is executed, the process proceeds to step ST23.

In step ST23, the computer 216 executes noise reduction processing using the one or plurality of processors. In an example embodiment, noise reduction processing is executed on the small opening signal. The reason will be described below.

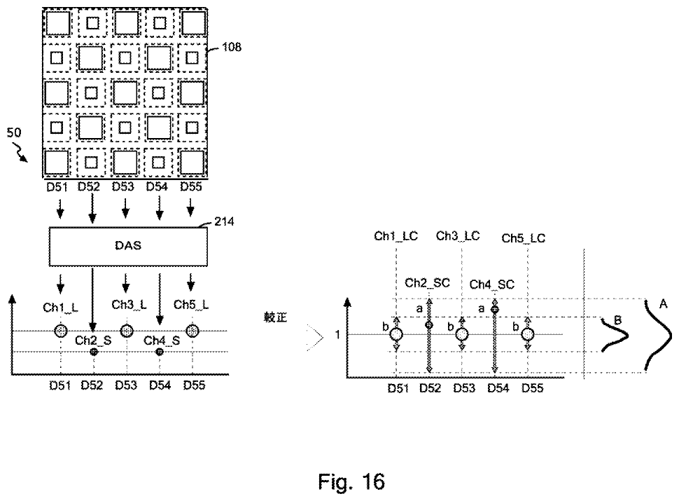

FIG. 16 is an explanatory diagram of a reason why noise reduction processing is executed on small opening signals. FIG. 16 depicts distributions A and B representing the uncertainty of the signals. Distribution A represents the uncertainty of the small opening signals, while distribution B represents the uncertainty of the large opening signals. In general, distributions A and B can be considered to be normal distributions. Furthermore, the range of uncertainty for distribution A is indicated by arrow a, and the range of uncertainty for distribution B is indicated by arrow b.

The large opening signal is obtained from X-rays passing through the large opening parts with large opening areas, and therefore, the utilization efficiency of the X-ray dose is considered to be high, and the range of uncertainty b is considered to be narrow.

In contrast, the small opening signals are signals obtained from X-rays that have passed through the small opening parts with small opening areas. Therefore, the utilization efficiency of the X-ray dose thereof is considered to be low, and the uncertainty range a is considered to be wider than the uncertainty range b of the large opening signals. Therefore, the small opening signals are susceptible to noise, resulting in reduced contrast resolution.

Thus, in an example, in step ST23, processing is executed to reduce the noise of the calibrated small opening signals on the basis of the calibrated large opening signals. Hereinafter, the noise reduction processing step ST23 will be described. Note that step ST23 includes steps ST231 to ST233, as depicted in FIG. 13. Each step will be described in order.

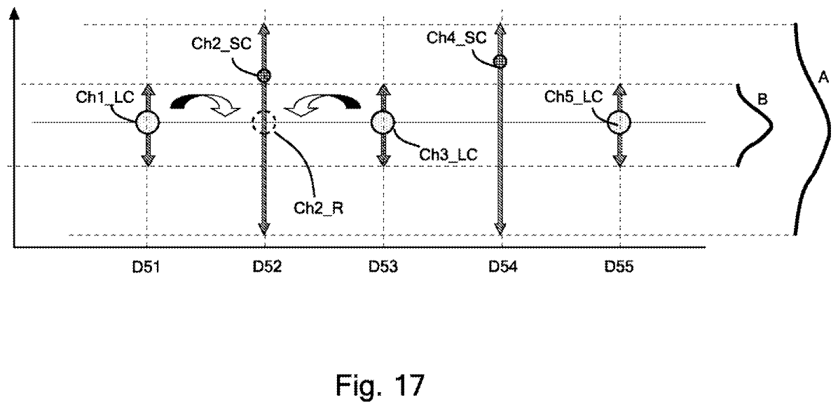

As previously described, the small opening signals generated by the detector elements have large variations due to noise (the width of uncertainty is large). Therefore, in the noise reduction processing, in step ST231, a reference signal is obtained as a basis for determining how much the small opening signals varied due to noise (see FIG. 17).

FIG. 17 is an explanatory diagram of a method for determining a reference signal for the small opening signals.

FIG. 17 depicts the calibrated signals (Ch1_LC, Ch2_SC, Ch3_LC, Ch4_SC, Ch5_LC). First, focusing on the calibrated small opening signal Ch2_SC of the detector element D52, it is considered that the reference signal for the small opening signal Ch2_SC is obtained. As described above, the large opening signals are signals with less variation due to noise than the small opening signals. Thus, in an example embodiment, a reference signal is generated on the basis of the large opening signals to determine the extent to which the small opening signals varied due to noise. With reference to FIG. 17, the signals of the detector elements D51 and D53 adjacent to the detector element D52 are the calibrated large opening signals Ch1_LC and Ch3_LC. Therefore, in an example, the computer 216 calculates, on the basis of the calibrated large opening signal Ch1_LC of the detector element D51 and the calibrated large opening signal Ch3_LC of the detector element D53, a reference signal Ch2_R that serves as a basis for determining the extent to which the calibrated small opening signal Ch2_SC varied due to noise. Specifically, on the basis of the calibrated large opening signals Ch1_LC and Ch3_LC, interpolation processing is executed to obtain an interpolation signal Ch2_R for the detector element D52, and the interpolation signal Ch2_R is used as the reference signal. After calculating the interpolation signal (reference signal) Ch2_R, the process proceeds to step ST232.

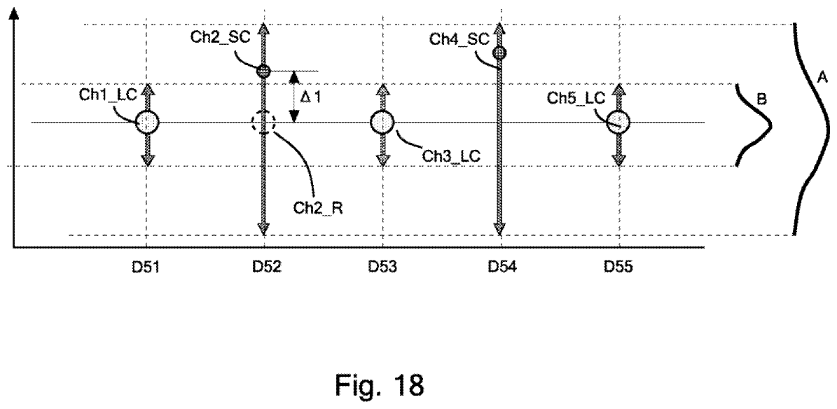

FIG. 18 is an explanatory diagram of step ST232. In step ST232, the computer 216 uses the one or plurality of processors to calculate a difference value Δ1 between the calibrated small opening signal Ch2_SC of the detector element D52 and the interpolation signal Ch2_R. After the difference value Δ1 is calculated, the process proceeds to step ST233.

### (3) Step ST233

In step ST233, the computer 216 uses the one or plurality of processors to correct the calibrated small opening signal Ch2_SC on the basis of the difference value Δ1 and to obtain the corrected small opening signal. Hereinafter, how the calibrated small opening signal Ch2_SC is corrected on the basis of the difference value Δ1 will be described. Note that in the following description, correction of the calibrated small opening signal Ch2_SC is divided into the following two:
(3A): Correction of the calibrated small opening signal Ch2_SC when the difference value Δ1 is large, and
(3B): Correction of the calibrated small opening signal Ch2_SC when the difference value Δ1 is small

As the difference value Δ1 increases, the likelihood of the calibrated small opening signal Ch2_SC varying due to noise decreases. Therefore, the calibrated small opening signal Ch2_SC is more likely to be the correct signal, while the interpolation signal Ch2_R is less likely to be the correct signal. Thus, when the difference value Δ1 is large, the weighting of the calibrated small opening signal Ch2_SC is increased while that of the interpolation signal Ch2_R is decreased, thereby correcting the calibrated small opening signal Ch2_SC.

As the difference value Δ1 decreases, the likelihood of the calibrated small opening signal Ch2_SC varying due to noise increases. Therefore, the calibrated small opening signal Ch2_SC is less likely to be the correct signal, while the interpolation signal Ch2_R is more likely to be the correct signal. Thus, when the difference value Δ1 is small, the weighting of the calibrated small opening signal Ch2_SC is decreased while that of the interpolation signal Ch2_R is increased, thereby correcting the calibrated small opening signal Ch2_SC.

Therefore, in an example, the calibrated small opening signal Ch2_SC is corrected in accordance with concepts (3A) and (3B) above. Specifically, the small opening signal is corrected in accordance with the following equation.

Chi_C=w×Chi_SC+(1−w)×Chi_R (1)

Herein, i represents the channel number, Chi_C represents the corrected small opening signal, w represents the weighting coefficient, Chi_SC represents the calibrated small opening signal, and Chi_R represents the interpolation signal

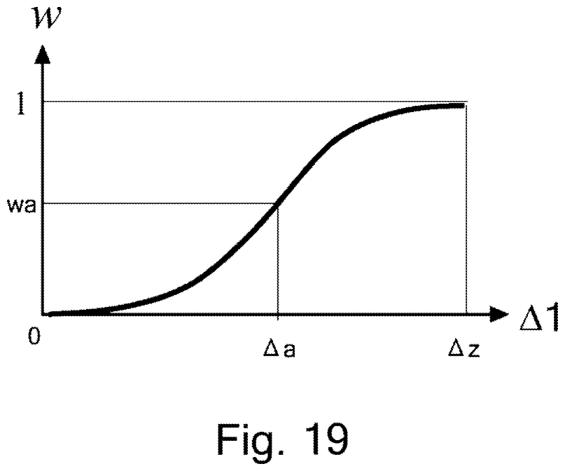

Note that the value of the weighting coefficient w is determined on the basis of the difference value Δ1. For example, the storing device stores a lookup table that defines the relationship between the weighting coefficient w and the difference value Δ1, and from this lookup table, the value of the weighting coefficient w can be determined on the basis of the difference value Δ1. FIG. 19 schematically depicts an example of the lookup table.

In FIG. 19, as the difference value Δ1 increases, the weighting coefficient w increases, and as the difference value Δ1 decreases, the weighting coefficient w decreases. Therefore, the larger the difference value Δ1, the greater the weighting of the calibrated small opening signal Chi_SC, and the smaller the weighting of the interpolation signal Chi_R. On the other hand, the smaller the difference value Δ1, the smaller the weighting of the calibrated small opening signal Chi_SC, and the greater the weighting of the interpolation signal Chi_R. Furthermore, on the basis of the weighted calibrated small opening signal Chi_SC and the weighted interpolation signal Chi_R, a corrected small opening signal Chi_C is obtained. Thus, in accordance with equation (1), it can be seen that the calibrated small opening signal can be corrected in accordance with concepts (3A) and (3B) above.

For example, in the case of the calibrated small opening signal Ch2_SC, i=2, and therefore, the corrected small opening signal Ch2_C can be calculated using equation (2) by substituting i=2 into equation (1).

Ch2_C=w×Ch2_SC+(1−w)×Ch2_R (2)

Furthermore, when Δ1=Δa, w=wa. Therefore, in this case, the corrected small opening signal Ch2_C can be calculated using the following equation.

Ch2_C=wa×Ch2_SC+(1−wa)×Ch2_R (3)

Furthermore, as depicted in FIG. 19, when Δ1=0, w=0. Therefore, the corrected small opening signal Ch2_C can be calculated using the following equation.

Ch2_C=Ch2_R (4)

On the other hand, as depicted in FIG. 19, when Δ1=Δz, w=1. Therefore, the corrected small opening signal Ch2_C can be calculated using the following equation.

Ch2_C=Ch2_SC (5)

In other words, when w=1, it can be seen that the calibrated small opening signal Ch2_SC is the correct signal.

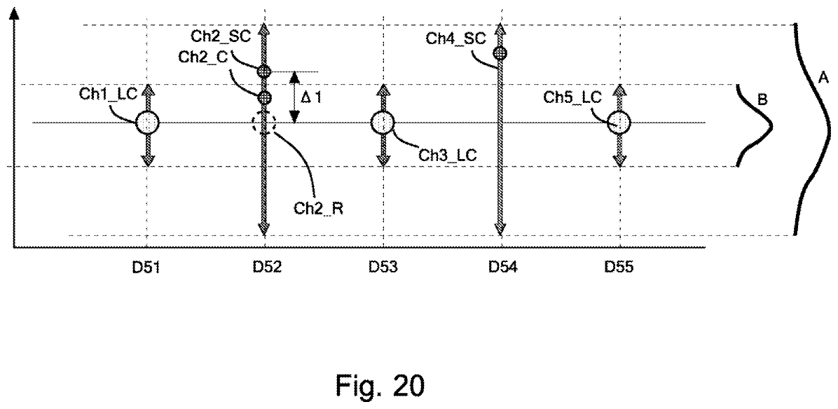

FIG. 20 depicts an example of the corrected small opening signal Ch2_C. FIG. 20 depicts the corrected small opening signal Ch2_C as an intermediate value between the calibrated small opening signal Ch2_SC and the interpolation signal Ch2_R.

Note the abovementioned example describes a correction method for channel number i when i=2 (i.e., the calibrated small opening signal Ch2_SC), but the corrected small opening signal can be calculated using equation (1) for other channel numbers as well. For example, when i=4 for the calibrated small opening signal Ch4_SC, the corrected small opening signal Ch4_C can be calculated using the following equation (6).

Ch4_C=w×Ch4_SC+(1−w)×Ch4_R (6)

Note that the interpolation signal Ch4_R in equation (6) can be calculated by interpolating between the calibrated large opening signals Ch3_LC and Ch5_LC. Therefore, all the calibrated small opening signals Chi_SC can be corrected by equation (1). After the calibrated small opening signals are corrected, the process proceeds to step ST24.

In step ST24, the computer 216 uses the one or plurality of processors to correct the large opening signals. The small opening signals are generated on the basis of X-rays that have passed through the small opening parts with small opening areas, and thus can have high spatial resolution. However, the large opening signals are generated on the basis of X-rays that passed through large opening parts with large opening areas, compared to the small opening signals, and thus have the problem in which the spatial resolution deteriorates. Thus, in step ST24, the calibrated large opening signal is corrected on the basis of the calibrated small opening signal such that the spatial resolution of the calibrated large opening signal is enhanced. Hereinafter, correction processing for the large opening signals will be described. Note that step ST24 includes steps ST241 to ST243, as depicted in FIG. 13. Each step will be described in order.

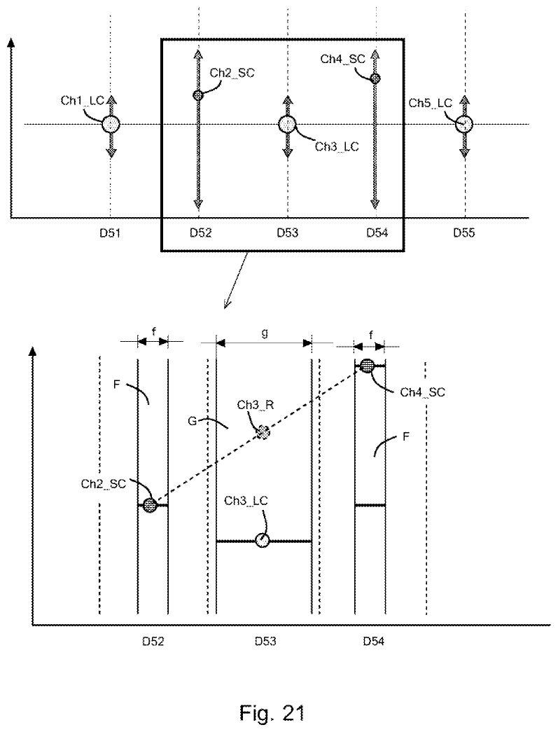

The detector elements receive X-rays that have passed through the large opening parts. Therefore, the large opening signals generated by the detector elements become signals with deteriorated spatial resolution. Thus, a reference signal is obtained as a reference to determine how much the spatial resolution of the large opening signals has deteriorated (see FIG. 21).

FIG. 21 is an explanatory diagram of a method for determining a reference signal for the large opening signals. Note that in FIG. 21, among the calibrated signals (Ch1_LC, Ch2_SC, Ch3_LC, Ch4_SC, Ch5_LC) depicted in FIG. 17, the calibrated signals (Ch2_SC, Ch3_LC, Ch4_SC) of the detector elements D52, D53, and D54 are depicted in an enlarged view. For convenience of explanation, elongated regions F and G representing differences in opening area are depicted for the detector elements D52, D53, and D54. Regions F and G have widths f and g, respectively. The detector elements D52 and D54 are depicted as regions F. The detector element D53 is an element with a large opening area, while the detector elements D52 and D54 are elements with small opening areas. Therefore, the width g of region G is wider than the width f of regions F.

First, focusing on the calibrated large opening signal Ch3_LC of the detector element D53, it is considered that the reference signal for the large opening signal Ch3_LC is obtained. As described above, the small opening signals are signals with higher spatial resolution than the large opening signals. Therefore, in an example, a reference signal is generated on the basis of the small opening signals to determine how much the spatial resolution of the large opening signals has deteriorated. With reference to FIG. 21, the signals of the detector elements D52 and D54 adjacent to the detector element D53 are the calibrated small opening signals Ch2_SC and Ch4_SC. Therefore, in an example, the computer 216 calculates, on the basis of the calibrated small opening signal Ch2_SC of the detector element D52 and the calibrated small opening signal Ch4_SC of the detector element D54, a reference signal Ch3_R that serves as a basis for determining the extent to which the spatial resolution of the calibrated large opening signal Ch3_LC has deteriorated. Specifically, on the basis of the calibrated small opening signals Ch2_SC and Ch4_SC, interpolation processing is executed to obtain an interpolation signal Ch3_R for the detector element D53, and the interpolation signal Ch3_R is used as the reference signal. After calculating the interpolation signal (reference signal) Ch3_R, the process proceeds to step ST242.

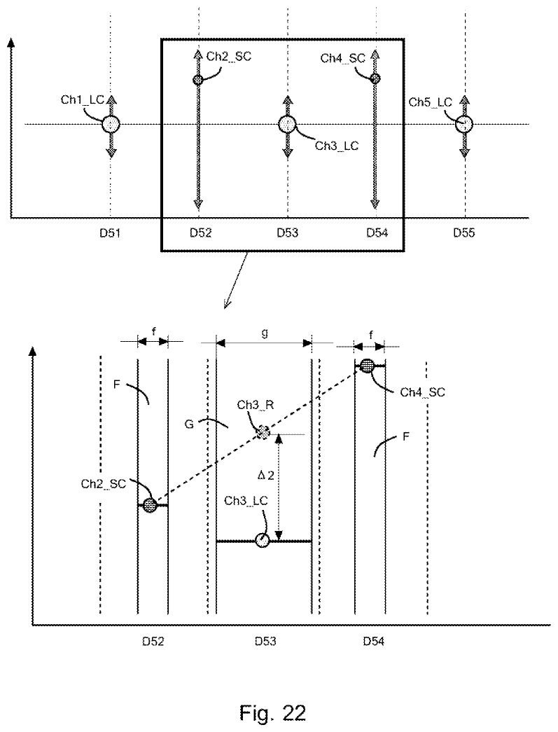

FIG. 22 is an explanatory diagram of step ST242. In step ST242, the computer 216 uses the one or plurality of processors to calculate a difference value Δ2 between the calibrated large opening signal Ch3_LC of the detector element D53 and the interpolation signal Ch3_R. The difference value Δ2 can be expressed by the following equation.

Δ2=Ch3_LC−Ch3_R (7)

After the difference value Δ2 is calculated, the process proceeds to step ST243.

In step ST243, the computer 216 uses the one or plurality of processors to correct the calibrated large opening signal Ch3_LC on the basis of the difference value Δ2 and to obtain the corrected large opening signal. Hereinafter, how the calibrated large opening signal Ch3_LC is corrected will be described.

Note that in explaining a correction method for the calibrated large opening signal Ch3_LC, the description will be divided into a case in which the difference value Δ2 is Δ2=0 and a case in which Δ2≠0.

In the case in which Δ2=0, the interpolation signal Ch3_R matches the calibrated large opening signal Ch3_LC. In this case, within the range of the width g of the detector element D53, the true value of the calibrated large opening signal Ch3_LC changes linearly.

### (2) When Difference Value Δ2≠0

In the case in which Δ2≠0, the interpolation signal Ch3_R deviates from the calibrated large opening signal Ch3_LC. In this case, within the range of the width g of the detector element D53, the true value of the calibrated large opening signal Ch3_LC changes non-linearly. As for the non-linear change of the true value of the calibrated large opening signal Ch3_LC, changes that are quadratic or more complex curves with more inflection points than quadratic changes can be considered. Herein, in order to facilitate understanding of the non-linear change in the true value of the calibrated large opening signal Ch3_LC, an example of non-linear change will be expressed by a quadratic curve to describe the non-linear change of the calibrated large opening signal Ch3_LC. Note that Δ2≠0 means that there are cases where Δ2<0 and cases where Δ2>0. Hereinafter, a description will be made dividing between cases of Δ2<0 and cases of Δ2>0.

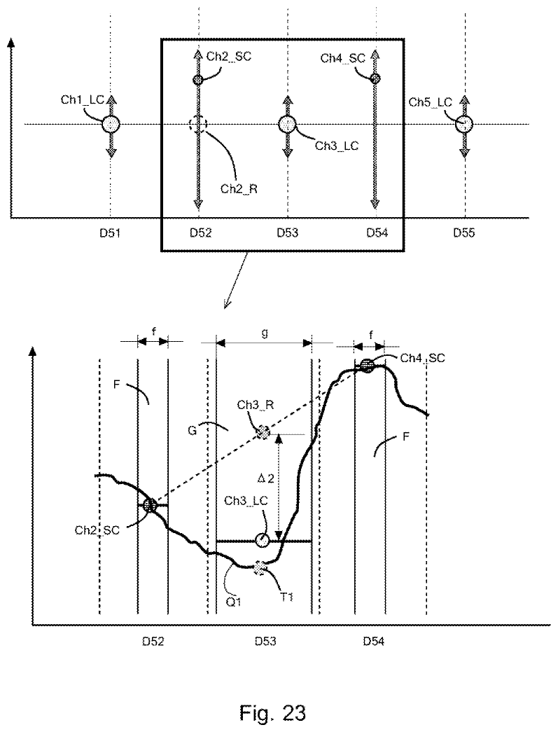

FIG. 23 is an explanatory diagram of the true value of a signal when Δ2<0. As previously described, Δ2 is expressed as Δ2=Ch3_LC−Ch3_R (see Equation 7). Therefore, in the case of Δ2<0, the calibrated large opening signal Ch3_LC is positioned below the interpolation signal Ch3_R. In this case, the true value of the calibrated large opening signal Ch3_LC within the range of the width g of the detector element D53 changes in accordance with a quadratic curve Q1 that is convex downward. Note that although the detector element D53 corresponds to the large opening part with a large opening area and generates the calibrated large opening signal Ch3_LC on the basis of the X-rays that pass through the large opening part, it is assumed herein that the detector element D53 corresponds to the small opening part with a small opening area and generates a signal on the basis of the X-rays that pass through the small opening part. In this case, if the signal of the detector element D53 obtained on the basis of the X-rays that passed through the small opening part is represented with the reference numeral “T1”, the signal T can be considered to correspond to a minimum value or a value near the minimum of the quadratic curve Q1. Therefore, it is possible to consider that the true value of the calibrated large opening signal Ch3_LC of the detector element D53 can be represented by the signal T1. Furthermore, the calibrated large opening signal Ch3_LC corresponds to the average value of the signal within the range of the detector element D53 in the direction of width g. Therefore, if Δ2<0, the condition holds that the true value T1 of the calibrated large opening signal Ch3_LC has a value smaller than that of the calibrated large opening signal Ch3_LC (is positioned below Ch3_LC).

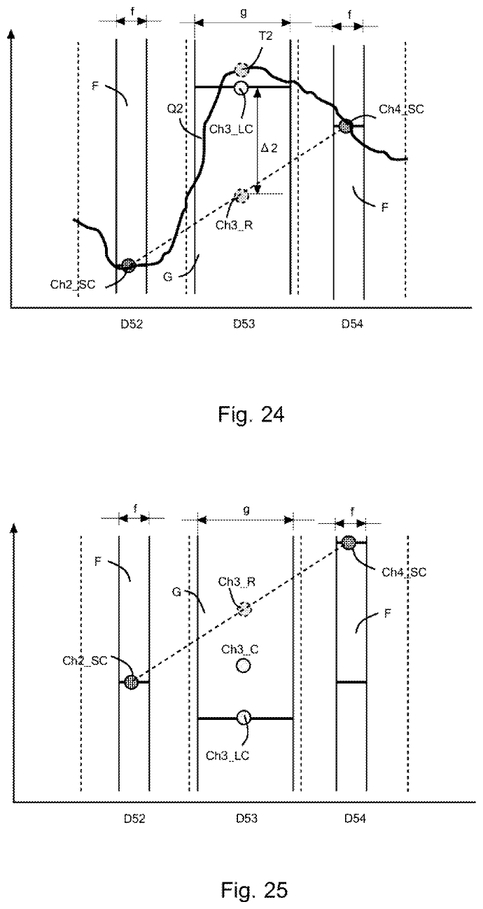

Next, the case of Δ2>0 will be described. FIG. 24 is an explanatory diagram of the true value of a signal when Δ2>0. In the case of Δ2>0, the calibrated large opening signal Ch3_LC is positioned above the interpolation signal Ch3_R. In this case, the true value of the signal within the range of the width g of the detector element D53 changes in accordance with a quadratic curve Q2 that is convex upward. Herein, it is assumed that the detector element D53 generates a signal on the basis of the X-rays that pass through the small opening part. In this case, if the signal of the detector element D53 obtained on the basis of the X-rays that passed through the small opening part is represented with the reference numeral “T2”, the signal T2 can be considered to correspond to a maximum value or a value near the maximum of the quadratic curve Q2. Therefore, it is possible to consider that the true value of the calibrated large opening signal Ch3_LC of the detector element D53 can be represented by the signal T2. Furthermore, the calibrated large opening signal Ch3_LC corresponds to the average value of the signal within the range of width g of the detector element D53. Therefore, if Δ2>0, the condition holds that the true value T2 of the calibrated large opening signal Ch3_LC has a value larger than that of the calibrated large opening signal Ch3_LC (is positioned above Ch3_LC).

Therefore, if the value of Δ2 is known, the relationship between the large opening signal Ch3_LC and the true value of the large opening signal Ch3_LC can be specified.

In a example, a correction function F(Δ2) incorporating the relationship above (relationship between the large opening signal Ch3_LC and the true value of the large opening signal Ch3_LC) is determined in advance according to the value of Δ2, and this correction function F(Δ2) is used to correct the large opening signal Ch2_LC. Specifically, the large opening signal is corrected in accordance with the following equation.

Chi_C=Chi_LC×F(Δ2) (8)

Herein, i represents the channel number, Chi_C represents the corrected large opening signal, Chi_LC represents the calibrated large opening signal, and F(Δ2) represents the correction function.

For example, in the case of the calibrated large opening signal Ch3_LC, i=3, and therefore, the corrected large opening signal Ch3_C can be calculated using equation (9) by substituting i=3 into equation (8).

Ch3_C=Ch3_LC×F(Δ2) (9)

FIG. 25 depicts an example of the corrected large opening signal Ch3_C. FIG. 25 depicts the corrected large opening signal Ch3_C as an intermediate value between the calibrated large opening signal Ch3_LC and the interpolation signal Ch3_R.

Note that in the abovementioned example, a correction method for channel number i when i=3 (i.e., the calibrated large opening signal Ch3_LC) was described, but the corrected large opening signal can be calculated using equation (8) for other channel numbers as well. Therefore, all the calibrated large opening signals Chi_LC can be corrected by equation (8). After the calibrated large opening signals are corrected, the process proceeds to step ST25 (see FIG. 13).

In step ST25, the image reconstructor 230 reconstructs a CT image 60 on the basis of the corrected small opening signal obtained in step ST23 and the corrected large opening signal obtained in step ST24. The corrected small opening signal has undergone noise reduction processing, and the corrected large opening signal has improved spatial resolution, making it possible to reconstruct the CT image 60 with both high spatial resolution and high contrast resolution.

As described above, in the example, the detector system 30 having the limiting member 20 is used to generate the small opening signal and the large opening signal. Furthermore, in step ST23, a small opening signal with reduced noise can be obtained by using the large opening signal to correct the small opening signal. Furthermore, in step ST24, by using the small opening signal to correct the large opening signal, a large opening signal with improved spatial resolution can be obtained. Therefore, a high-quality CT image 60 that simultaneously achieves high spatial resolution and high contrast resolution can be reconstructed.

Furthermore, a deep learning-based technique can be used for the noise reduction processing in step ST23 and for the processing to enhance the spatial resolution in step ST24.

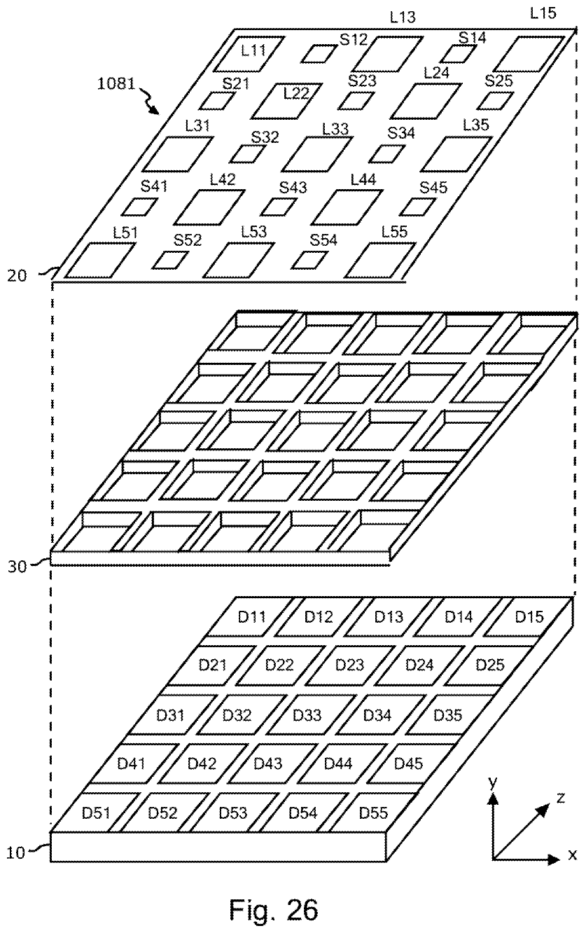

Note that in the abovementioned detector assembly 108, the limiting member 20 is disposed between the detector 10 and the collimator 30. However, the limiting member 20 does not necessarily have to be provided between the detector 10 and the collimator 30 and may be provided above the collimator 30. FIG. 26 is a diagram depicting an example of a detector assembly 1081 in which the limiting member 20 is disposed above the collimator 30. In FIG. 26, the limiting member 20 is positioned on the opposite side of the detector 10 from the collimator 30. In other words, when viewed from the X-ray tube 104 towards the detector assembly 1081, the limiting member 20, collimator 30, and detector 10 are superimposed in that order. Thus, the limiting member 20 may be in a position closer to the X-ray tube 104 than the collimator 30.

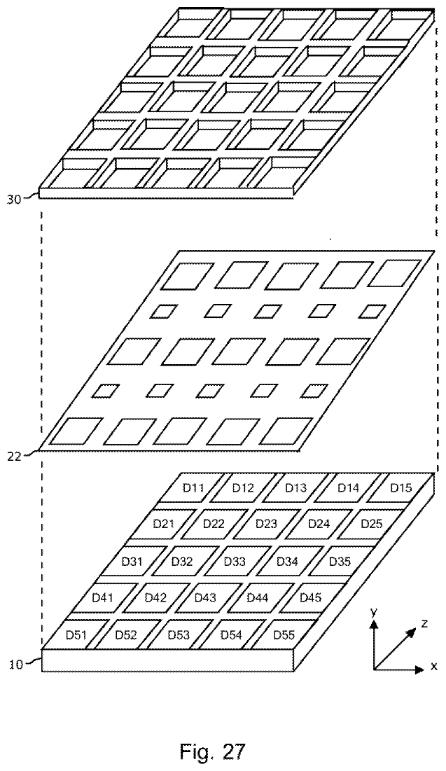

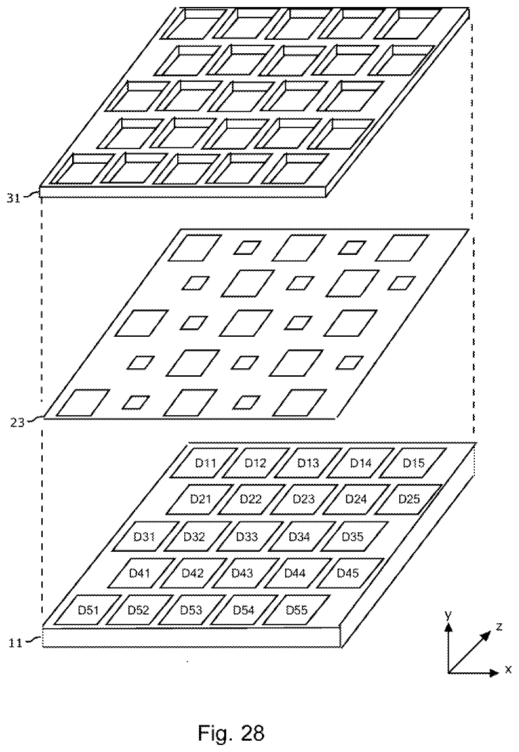

Furthermore, in the abovementioned limiting member 20, the large opening part and the small opening part are arranged alternately in the channel direction and the body axis direction (see FIG. 6 and FIG. 26). However, so long as the limiting member 20 includes a large opening part and a small opening part, various modified examples of the limiting member can be considered. For example, in the modified example depicted in FIG. 27, a limiting member 22 is configured such that a row only having small opening parts and a row only having large opening parts are provided in the channel direction, and the small opening parts and large opening parts are arranged alternately only in the body axis direction (z direction). Furthermore, FIG. 28 depicts an example in which the detector elements of the detector 11 are offset by a prescribed length in the body axis direction or channel direction with respect to an adjacent detector element, with the collimator 31 also being offset. In this case, a limiting member 23 can be provided such that each opening part (small opening part and/or large opening part) is offset by a prescribed length in the body axis direction or channel direction with respect to an adjacent opening part.

Furthermore, in the abovementioned embodiment, a large opening part and a small opening part are disclosed as examples of opening parts with different opening areas. However, if the limiting member 20 includes opening parts with different opening areas, the limiting member 20 may include three or more types of opening parts with different opening areas (e.g., small opening parts, medium opening parts, and large opening parts).

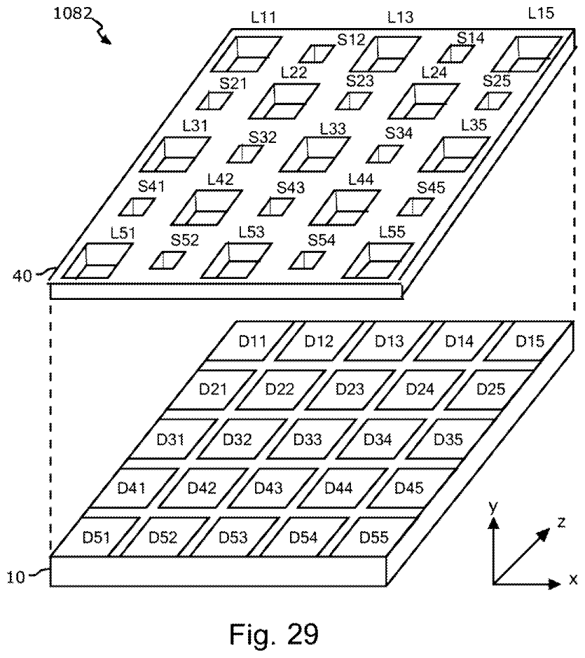

Furthermore, in the abovementioned embodiment, the limiting member 20 constitutes a member different from the collimator 30. However, instead of configuring the limiting member 20 and the collimator 30 as separate members, an integrated member having both the functions of the limiting member 20 and the collimator 30 may be configured. FIG. 29 is a diagram depicting an example of the detector assembly 1082 including a member 40 having both the functions of a limiting member and a collimator. The member 40 is configured to be thicker than the limiting member 20 (e.g., see FIG. 6) so as to be able to have the function of a collimator. Since the member 40 performs both the functions of a limiting member and a collimator, a dedicated collimator member is not required, which can reduce the number of parts used in the detector assembly 1082.

Note that in Embodiments 1 and 2, examples are depicted in which the limiting member is applied to the CT system 100. However, the present invention is not limited to the CT system 100, and the limiting member can be applied to a system other than the CT system 100 (e.g., PET-CT system), so long as the medical system irradiates X-rays onto the subject.

Note that the interpolation processing executed in Embodiments 1 and 2 can utilize various techniques. For example, techniques based on mathematical models, techniques using deep learning, and the like can be employed.

1. A detector system, comprising:
a detector having a plurality of detector elements for detecting X-rays irradiated from an X-ray tube; and
a member disposed between the X-ray tube and the detector and having a plurality of opening parts corresponding to the plurality of detector elements, the plurality of opening parts being opening parts through which the X-rays pass, and some of the plurality of opening parts having opening areas different from the remaining opening parts.

2. The detector system according to claim 1, wherein the member has a plurality of first opening parts having a first opening area and a plurality of second opening parts having a second opening area, and
the plurality of first opening parts and the plurality of second opening parts are arranged such that each of the first opening parts has at least two second opening parts adjacent thereto.

3. The detector system according to claim 2, wherein the second opening parts are disposed on both sides of each of the first opening parts in a channel direction, and
the second opening parts are disposed on both sides of each of the first opening parts in a body axis direction.

4. A CT system, comprising a detector system that includes:
a detector having a plurality of detector elements for detecting X-rays irradiated from an X-ray tube; and
a member disposed between the X-ray tube and the detector and having a plurality of opening parts corresponding to the plurality of detector elements, the plurality of opening parts being opening parts through which the X-rays pass, and some of the plurality of opening parts having opening areas different from the remaining opening parts,
the detector system being configured so as to generate a signal corresponding to each of the detector elements, wherein
a CT image is reconstructed on the basis of the signal corresponding to each of the detector elements.

5. The CT system according to claim 4, wherein
the member has a plurality of first opening parts having a first opening area and a plurality of second opening parts having a second opening area,
each of the first opening parts is a large opening part having a larger opening area than each of the second opening parts, and
each of the second opening parts is a small opening part having a smaller opening area than each of the first opening parts.

6. The CT system according to claim 5, wherein
the detector includes a plurality of first detector elements corresponding to a plurality of small opening parts and a plurality of second detector elements corresponding to a plurality of large opening parts, and
the detector system uses the first detector element corresponding to the small opening part to generate a small opening signal that represents a signal corresponding to the first detector element, and uses the second detector element corresponding to the large opening part to generate a large opening signal that represents a signal corresponding to the second detector element.

7. The CT system according to claim 6, the CT system using one or a plurality of processors to execute the following:
generating, from the plurality of small opening signals and the plurality of large opening signals generated by the detector system, first projection data including a plurality of the large opening signals and second projection data including a plurality of the small opening signals;
executing interpolation processing on the first projection data to interpolate a data point of the first detector element; and
executing interpolation processing on the second projection data to interpolate a data point of the second detector element.

8. The CT system according to claim 7, the CT system using one or a plurality of processors to execute the following:
reconstructing the first CT image on the basis of the interpolated first projection data; and
reconstructing the second CT image on the basis of the interpolated second projection data, wherein
the first CT image is a high contrast resolution CT image and the second CT image is a high spatial resolution CT image.

9. The CT system according to claim 7, the CT system using one or a plurality of processors to:
execute data blending processing for combining the interpolated first projection data and the interpolated second projection data; and generate the first CT image and the second CT image on the basis of data obtained by the data blending processing.

10. The CT system according to claim 6, wherein
calibration data is stored in a storing device,
the CT system using one or a plurality of processors to execute the following:
calibrating a large opening signal and a small opening signal on the basis of the calibration data; and
reducing noise in the calibrated small opening signal on the basis of a calibrated large opening signal.

11. The CT system according to claim 10, the CT system using one or a plurality of processors to execute the following:
executing interpolation processing on the basis of a plurality of calibrated large opening signals to obtain a first interpolation signal for the first detector element;
calculating a first difference value between a calibrated small opening signal of the first detector element and the first interpolation signal; and
correcting the calibrated small opening signal of the first detector element on the basis of the first difference value.

12. The CT system according to claim 11, the CT system using one or a plurality of processors to execute the following:
determining a weighting coefficient value on the basis of the first difference value; and
weighting the calibrated small opening signal of the first detector element and the first interpolation signal on the basis of the weighting coefficient value, and correcting the calibrated small opening signal on the basis of the weighted and calibrated small opening signal and the weighted first interpolation signal.

13. The CT system according to claim 12, the CT system using one or a plurality of processors to:
execute weighting of the calibrated small opening signal and the first interpolation signal such that, as the first difference value increases, the weighting of the calibrated small opening signal increases and the weighting of the first interpolation signal decreases; and
execute weighting of the calibrated small opening signal and the first interpolation signal such that, as the first difference value decreases, the weighting of the calibrated small opening signal decreases and the weighting of the first interpolation signal increases.

14. The CT system according to claim 10, the CT system using one or a plurality of processors to:
correct the calibrated large opening signal on the basis of the calibrated small opening signal such that the spatial resolution of the calibrated large opening signal is enhanced.

15. The CT device according to claim 14, the CT system using one or a plurality of processors to execute the following:
executing interpolation processing on the basis of a plurality of calibrated small opening signals to obtain a second interpolation signal for the second detector element;
calculating a second difference value between a calibrated large opening signal of the second detector element and the second interpolation signal of the second detector element; and
correcting the calibrated large opening signal of the second detector element on the basis of the second difference value.

16. The CT device according to claim 15, the CT system using one or a plurality of processors to execute the following:
correcting the calibrated large opening signal under the condition that, if the second difference value is a negative value, the true value of the calibrated large opening signal has a value that is smaller than a signal value of the calibrated large opening signal; and
correcting the calibrated large opening signal under the condition that, if the second difference value is a positive value, the true value of the calibrated large opening signal has a value that is larger than the signal value of the calibrated large opening signal.
